(19) United States
(12) Patent Application Publication
(10) Pub. No.: US 2013/0303836 A1
(43) Pub. Date: Nov. 14, 2013

(54) Recreational, Occupational, And Physical Therapy System and Apparatus

(71) Applicant: Kurt W. Coble, Mamaroneck, NY (US)

(72) Inventor: Kurt W. Coble, Mamaroneck, NY (US)

(21) Appl. No.: 13/889,378

(22) Filed: May 8, 2013

Related U.S. Application Data
(60) Provisional application No. 61/643,925, filed on May 8, 2012.

(51) Int. Cl. A61M 21/02

(52) U.S. Cl. CPC A61M 21/02
USPC 600/28

(57) ABSTRACT

The present invention uses the power of music to provide both feedback and motivation to physical, recreational, occupational therapy patients to enable music expression, therapeutic engagement and compliance.

### REFERENCE TO RELATED APPLICATION

Provisional application No. 61/643,925, filed on May 8, 2012. Publication Classification U.S. Cl. A61H 2201/00

### BACKGROUND

Recreational, occupational, and physical therapy are often a required part of the process of healing after an accident or injury. Most commonly, a therapist will guide the patient through a series of motions designed to build strength in muscles to prevent the loss of mobility before it occurs by developing fitness and wellness-oriented programs for healthier and more active lifestyles, providing services to individuals and populations to develop, maintain and restore maximum movement and functional ability throughout the lifespan. This includes providing services in circumstances where movement and function are threatened by aging, injury, disease or environmental factors. Functional movement is central to what it means to be healthy.

In other cases, recreational, occupational, and physical therapy aids patients who suffer from neurological, physical, or developmental delays which limit their ability to control their bodies and therefore leave them with reduced functional movement. The 2010 Census shows that 56.8 million individuals in the US are living with some form of disability. For these patients the standard techniques of applying guided motions are often both fruitless and frustrating. Their lack of control obstructs their capacity to perform the guided motions that would benefit both their functional movement and their general well-being.

Several recent inventions teach improvements to physical therapy equipment and methods. For example in U.S. Pat. Appl. No. 2007/0135738 by Bonutti et al. the inventor teaches the attachment of transducers to the physical therapy apparatus. These transducers measure the positions that the equipment has reached during a therapy session and saves the data to a computer. The therapist can review the data at a later time and monitor the patient's progress. In another example, U.S. Pat. Appl. No. Y2010/0113982 by Smith, M. teaches an article, such as an armband, that is worn by the patient during physical therapy. The article contains accelerometers which measure the position of the limb and communicate that data to a computer via a cable. As with the previous invention, the therapist can review the data at a later time and monitor the patient's progress. However, neither invention provides real time feedback to the patient, and neither invention can assist the patient with neurological, physical, or developmental delays achieve their therapy goals.

A need therefore exists for an improved physical therapy system that provides real time feedback and motivation to the patient and can be successfully adapted to work with patients having varying levels of neurological, physical, or developmental disabilities to enable therapeutic engagement and compliance.

### BRIEF SUMMARY OF THE INVENTION

The present invention uses the power of music to provide both feedback and motivation to physical, recreational, occupational therapy patients to enable music expression, therapeutic engagement and compliance. The patient interacts with the apparatus through various contact and non-contact sensors such as optical, mechanical, or motion sensors as they perform their guided motions. The sensors send their output to a controller which actuates one or more transducers that play a musical instrument.

The sensors send their output to a controller that actuates one or more transducers to play a musical instrument combining entertainment and musical expression with physical therapy.

The system can be successfully adapted to work with patients who have different levels of neurological, physical, or developmental disabilities to enable therapeutic engagement and compliance. Compliance can be measured with additional time-based recording device, but the primary measure of success is in the performance, mastery, and improvement of technique.

### BRIEF DESCRIPTION OF THE DRAWINGS

FIG. 1 is an image of an actuator used to play a musical instrument such as a percussion instrument.

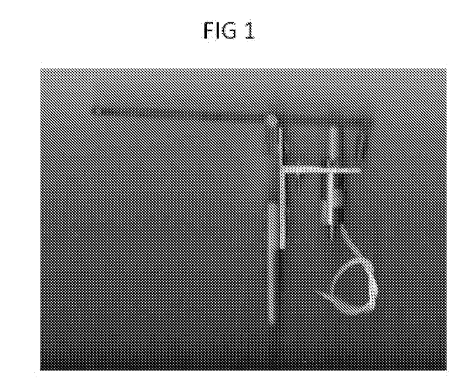

### DETAILED DESCRIPTION OF THE INVENTION

In his book “Musicophilia: Tales of Music and the Brain” (Oliver Sacks, Vintage 2008), Dr. Oliver Sacks, a neurologist, describes the integration of music in the deep structures of the brain. It is not uncommon to find people with profound neuro-developmental disabilities yet who retain the ability to understand, enjoy, and sometimes play music. Other researchers have noted improvements in patient compliance, cognition, and emotion that accompanies their caregiver singing to and/or with them.

The present invention uses the power of music to provide both feedback and motivation to physical, recreational, occupational therapy patients. The apparatus comprises a sensor, a controller, and a music source.

The sensor can comprise a non-contact sensor such as an optical sensor, a motion sensor, or an audio or voice activated sensor. The optical sensor may comprise a sensor that is activated when a beam of light is directed at the sensor, or may be activated when a beam of light that is already directed at the sensor is obstructed, interrupted, or otherwise broken. In one embodiment of the present invention an optical sensor is triggered when the guided motion of a limb breaks the light-path of the prepositioned light source. In another embodiment, the light source is attached to an article of clothing which triggers the optical sensor when the guided motion results in the light source illuminating the optical sensor.

The sensor can also comprise a motion sensor. Visible light, infrared light, or laser technology may be used for optical motion detection. Motion detection devices, such as PIR motion detectors, have a sensor that detects a disturbance in the infrared spectrum, such as a person's limb. Once detected, an electronic signal can be generated. A simple algorithm for motion detection by a fixed camera compares the current image with a reference image and simply counts the number of different pixels. Since images will naturally differ due to factors such as varying lighting, camera flicker, and CCD dark currents, pre-processing is useful to reduce the number of false positive alarms. One embodiment of the present invention comprises an infrared motion detector that is triggered when the guided motion of a limb disturbs the background infrared spectrum. Another embodiment comprises a fixed camera that is triggered when the guided motion of a limb causes the camera's current image to differ from the camera's reference image.

The sensor can also comprise a contact sensor such as a button, a toggle switch, a paddle switch, a musical keyboard key, a slider, a joystick, a touch pad, or a dial. The contact sensors can be used singly or in an array with different sensors being used to trigger different sounds or musical instruments. Some types of contact sensors, such as a button or toggle switch, may be used to trigger a single sound or action. Other types of contact sensors, such as a slider, joystick, or touch pad, may be used to trigger sounds or actions of adjustable duration or changing pitch. The contact sensor can be positioned such that when the guided motion of the limb has reached its desired extent, the contact sensor is touched.

One aspect of the invention is that the sensor is customized to accommodate the patient's abilities. In one embodiment, a quadriplegic patient may be fitted with a hat having a light source. The patient would use it to interact with an optical sensor. In another embodiment of the present invention a button is positioned so that it is contacted at the full extent of the guided motion of a limb, triggering a sound that provides feedback to the patient. In another embodiment the guided motion of a limb runs along a touch pad that triggers a tone of increasing frequency.

The controller can comprise a mechanical, pneumatic, electrical, electronic, or computerized device. The output signal of the contact or non-contact sensor initiates the operation of the controller. The controller actuates the music source when it receives a signal from the sensor. The input signal from the sensor can be via a wired or wireless connection. The controller may initiate a servo-motor which plays a musical instrument, an audio sequence on a MIDI device, or a pre-recorded tone or musical passage.

In one embodiment, the controller receives the sensor signal and an input audio signal and modifies a property of the input audio signal dependent on the sensor signal to obtain an output audio signal. In another embodiment, a function may map the values of the sensor signal to control parameters for modifying the property (or the properties) of the input audio signal. This function may be any monotonic function including a piecewise function characterized by a threshold value. In an embodiment, the controller only supplies the output audio signal to the music source if the associated sense signal indicates that a property of the light is above a particular threshold.

The controller processes the input audio signal into an output audio signal in accordance with the sensed property or the sensed properties. In an embodiment, the input audio signal is supplied to the music source if a particular property of the light is detected to be present and no audio signal is supplied to the music source if this particular property is not present. For example, the audio signal is only supplied to the music source when the intensity of the sensed light is above a particular threshold value.

As an example, the controller may comprise a voltage comparator. A comparator is a device that compares two voltages or currents and switches its output to indicate which is larger. A dedicated voltage comparator chip such as LM339 is designed to interface with a digital logic interface (to a TTL or a CMOS). The output is a binary state often used to interface real world signals to digital circuitry (see analog to digital converter). If there is a fixed voltage source from, for example, a DC adjustable device in the signal path, a comparator is just the equivalent of a cascade of amplifiers. When the voltages are nearly equal, the output voltage will not fall into one of the logic levels, thus analog signals will enter the digital domain with unpredictable results. To make this range as small as possible, the amplifier cascade is high gain. The circuit consists of mainly Bipolar transistors except perhaps in the beginning stage which will likely be field effect transistors. For very high frequencies, the input impedance of the stages is low. This reduces the saturation of the slow, large P-N junction bipolar transistors that would otherwise lead to long recovery times. Fast small Schottky diodes, like those found in binary logic designs, improve the performance significantly though the performance still lags that of circuits with amplifiers using analog signals. Slew rate has no meaning for these devices. For applications in flash ADCs the distributed signal across 8 ports matches the voltage and current gain after each amplifier, and resistors then behave as level-shifters.

The LM339 accomplishes this with an open collector output. When the inverting input is at a higher voltage than the non inverting input, the output of the comparator connects to the negative power supply. When the non inverting input is higher than the inverting input, the output is ‘floating’ (has a very high impedance to ground).”

The music source may comprise a musical instrument, a MIDI device, or a pre-recorded tone or musical passage. The musical instrument may comprise a drum, cymbal, tambourine, maracas, or other percussion instrument. In the present invention percussion instruments are played by means of a solenoid, motor or servo-motor, which is actuated by the controller. The motor powers a drumstick or other contact means, which moves to tap the percussion instrument when activated by the controller. The musical instrument may further comprise a guitar, mandolin, ukulele, bass, violin, or other stringed instrument. In the present invention stringed instruments are played by means of a motor or servo-motor, which is actuated by the controller. The motor powers an arm holding a plectrum or other contact means, which moves to strum or bow the stringed instrument when activated by the controller. The musical instrument may further comprise a trumpet, bugle, trombone, or other brass instrument or a flute, clarinet, saxophone, or other woodwind instrument. In the present invention brass or woodwind instruments are played by means of a pneumatic power source, which is actuated by the controller. The pressurized air is directed into the brass or woodwind instrument when activated by the controller. In addition any of the instruments in the present invention may be played by actuators that are powered electrically, hydraulically, or pneumatically.

As an example, an actuator device may be used to hit a drum. The actuator device may comprise a post and arm powered by a solenoid. Once the solenoid's electromagnet is energized by the controller, a plunger is forced upward coming in contact with a lever. The lever in turns moves in the opposite direction with a force equal to the force of the energized solenoid. The lever is constructed from a beam attached to ground by a hinge, or fulcrum.

The music device may comprise a MIDI device such as a synthesizer. In one embodiment of the present invention the controller may initiate a sequence in the synthesizer to produce musical notes or a preprogrammed musical passage. In another embodiment the controller may initiate a sequence of beats to guide the timing of the physical therapy. In yet another embodiment, multiple sensors may provide input to the controller or the musical device to allow multiple patients to play ensemble. For example, two or more sensors may be connected to the controller, with each sensor actuating a different instrument or MIDI sequences. In another embodiment, the different MIDI sequences or instrument tones may be connected to the controller by a means such as a memory card, a dongle, or USB drive, so that each patient could have their own musical instrument sounds. In this example, one patient could plug into the controller a USB drive containing a cello sound file, while a second patient plugs into the same controller a second USB dive with flute sound files, so that the two patients could play together.

In a physical therapy session the guided motions are replicated in a series to strengthen the muscles and increase the range of functional movement. In another embodiment a musical passage is broken into segments and the segments are played in sequence with the reps of the guided motions, so that the entire musical passage is played through the series of motions that make up the physical therapy exercise.

In another embodiment, a group of patients receiving physical therapy together can play a group of musical instruments ensemble. In yet another embodiment, customized musical arrangements may be written to accommodate the abilities of the members of the ensemble.

While the present invention can be used to treat recreational, occupational, and physical therapy patients in a therapeutic setting, it can also be used in a home setting, or in a recreational setting.

### EXAMPLES

### Example 1

In a physical therapy setting, an optical sensor is positioned so that the full extension of the patient's arm breaks a light beam. The optical sensor produces a signal that triggers an electronic controller to initiate a pleasing synthesized tone. The tone provides feedback to the patient that her arm has reached the desired position and extension.

### Example 2

A quadriplegic patient who can only move his head is fitted with a light source attached to a hat. He aims the light source at an array of optical sensors each of which is connected via a controller to a different drum in a drum kit. By exercising the muscles of his head and neck the patient can play the drum kit.

### Example 3

In a physical therapy setting, an button sensor is positioned so that the full extension of the patient's arm touches the button. The button sensor produces a signal that triggers an electronic controller to initiate a rhythm. The rhythm provides feedback to the patient to keep her guided motions in time.

### Example 4

In a recreational therapy setting group of patients with developmental delays that cause them to have poor control of their limbs are each fitted with a light source attached to a hat. They aim the light source at an optical sensor positioned in front of each of patient, each sensor being connected to a common controller each having USB drives with different musical instrument sound file. By exercising the guided motions of their heads and necks the patients can play their instruments ensemble.

### Example 5

In a recreational therapy setting a patient uses a group of dial and lever sensors that can be calibrated to produce a signal to control musical devices such as a guitar or violin resulting in a theraputic benefit.

### Example 6

In a occupational therapy setting sensors and light sources are attached to the patient's arms, hands, legs, feet, neck, head, or torso for the purpose of producing well regulated motions, which when properly completed will result in a desired musical tone or sequence, and thereby producing a therapeutic benefit.

I claim:

1. A physical, occupational, or recreational therapy apparatus comprising a sensor, a controller, and a music source.

2. The apparatus of claim 1 wherein the sensor comprises a contact sensor or a non-contact sensor.

3. The apparatus of claim 2 wherein the sensor comprises an optical sensor, a motion sensor, or an audio or voice activated sensor.

4. The apparatus of claim 1 wherein the controller comprises a mechanical, pneumatic, electrical, electronic, or computerized device.

5. The apparatus of claim 1 wherein the music source comprises a musical instrument, a MIDI device, or a pre-recorded tone or musical passage.

6. A physical, occupational, or recreational therapy apparatus comprising a sensor, a controller, and a music source wherein the apparatus provides feedback to the patient.

7. The apparatus of claim 6 wherein the feedback comprises musical notes, tones, or passages.

8. The apparatus of claim 6 wherein the feedback comprises information that the patient has reached the desired position of the guided motion.

9. The apparatus of claim 6 wherein the feedback comprises information that the patient has reached the desired duration of the guided motion.

10. The apparatus of claim 6 wherein the feedback comprises information that the patient has reached the desired rhythm of the guided motion.

11. A physical, occupational, or recreational therapy apparatus comprising one or more sensors, a controller, and a music source wherein multiple patients can connect to a common controller wherein the one or more sensors is selected from the list consisting of optical sensors, motion sensors, audio or voice activated sensors, buttons, toggle switches, paddle switches, sliders, joysticks, touch pads, and dials.

12. The apparatus of claim 11 wherein the controller can access multiple instrument sound files.

13. The apparatus of claim 12 wherein each of the instrument sound files is contained on a memory device connected to the controller.

14. The apparatus of claim 13 wherein the sensors are connected to the controller by wires or wirelessly.
